(19) United States
(12) Patent Application Publication
(10) Pub. No.: US 2026/0306734 A1
(43) Pub. Date: Oct. 8, 2026

(54) PACKAGING, STORAGE, AND PROTECTIVE TECHNIQUES FOR MEDICAL SYRINGES AND FLUID PATH COMPONENTS

(71) Applicant: BAYER HEALTHCARE LLC, Whippany, NJ (US)

(72) Inventors: David BERRY, Kittanning, PA (US); Sean Kelly MCKILLOP, Pittsburgh, PA (US); Thomas KINST, Pittsburgh, PA (US); Steven STRICKO, Trafford, PA (US)

(21) Appl. No.: 19/481,337

(22) PCT Filed: May 9, 2024
(86) PCT No.: PCT/US2024/028514

§ 371 Date: Nov. 4, 2025

Related U.S. Application Data
(60) Provisional application No. 63/465,342, filed on May 10, 2023.

(51) Int. Cl. A61M 5/00; A61L 2/08; A61L 103/15

(52) U.S. Cl. CPC A61M 5/002; A61L 2/08; A61L 2103/15; A61M 2209/06

(57) ABSTRACT

A syringe packaging system and associated methods are described. The syringe packaging system may include a dividing wall arrangement defining a plurality of spaces; and a container configured to receive the dividing wall arrangement and a plurality of syringes therein. At least a portion of the plurality of spaces may be configured to receive individual syringes of the plurality of syringes therein. The syringes are received in an upright, substantially parallel configuration and the dividing wall arrangement divides each individual syringe from one or more adjacent syringes. The syringe packaging system allows for reduced medical waste, improved recyclability, and a minimized carbon footprint. Methods for sterilizing a plurality of syringes contained in a packaging system is disclosed.

### CROSS-REFERENCE TO RELATED APPLICATIONS

This application claims priority to United States Provisional Patent Application Ser. No. 63/465,342, titled “Packaging, Storage, and Protective Techniques for Medical Syringes and Fluid Path Components” and filed on 10 May 2023, the disclosure of which is hereby incorporated by reference in its entirety.

### BACKGROUND

### Technical Field

The present disclosure relates to packaging systems, storage solutions, and protective techniques and structures for storage, sterilization, and shipment of medical devices. In certain aspects, these medical devices more particularly include syringes, fluid path components, and medical injectors which are useful in a variety of medical procedures and tests.

### Description of Related Art

Syringes and medical injectors designed for containing fluid such as contrast media and saline are commonly used to introduce fluid into a patient's body in a wide variety of medical diagnostic imaging procedures. These medical devices need to be manufactured, packaged, and delivered to customers such as hospitals, medical practitioners, and other health care providers in a manner that promotes sterility and structural integrity and retains the devices in proper functioning condition. Previously employed syringe packaging systems and storage solutions could benefit from improvements which more effectively address these important sterility, durability, recyclability, and operability considerations, among other factors.

Packaging for syringes must comply with applicable sterility, durability, and labeling standards in connection with delivering the syringes to an end user or customer. The syringe packaging system must be sufficiently resistant to breaches of sterility and infiltration of foreign particulates into the medical device or the package. Further, the syringes and fluid path components are typically sterilized after packaging, either in the individual package or in bulk packaging containing multiple injector components. Sterilization techniques may involve exposure of the packaged components to irradiation with electron beam or exposure to ethylene oxide. Further, in association with storage and delivery of syringes, a packaging system needs to resist scratches, slices, or other physical damage to the most important structural parts of the syringes and to resist contamination of the sterile fluid paths within the syringes. In addition, the syringe packaging system should be capable of being conveniently stacked or stored with respect to containers used for shipping products to customers. Also, the structural design of the packaging system preferably should be compatible with the shelves or other storage areas typically used for storage of medical devices at the end user's facility, such as the imaging suite. Also, syringe packaging systems and solutions are needed which require few or substantially no special modifications to the underlying structural design and function of the syringe itself.

In addition, conventional packaging systems utilize materials that are not readily recyclable or are not manufactured from sustainable sources. Thus, the carbon footprint associated with the packaging design and/or materials is increased.

In view of the above-described issues, enhanced techniques and solutions are needed for medical device packaging systems, particularly for packaging, shipment, and storage of medical syringes.

### BRIEF SUMMARY

In some embodiments, a syringe packaging system may include a dividing wall arrangement defining a plurality of spaces; and a container configured to receive the dividing wall arrangement and a plurality of syringes therein. At least a portion of the plurality of spaces may be configured to receive an individual syringe of the plurality of syringes therein. The plurality of syringes may be received in an upright, substantially parallel configuration and the dividing wall arrangement may divide each individual syringe from one or more adjacent syringes.

In some embodiments, the syringe packaging system may further include a cover defining a plurality of openings, with each opening of the plurality of openings being configured to receive a distal end of a syringe of the plurality of syringes therethrough. Each of the plurality of openings may include at least one slit circumferentially arranged around a periphery of each of the plurality of openings. Each of the plurality of syringes may include a clip feature located on the distal end, and each of the plurality of openings may be configured to receive the clip feature therethrough. The cover may include a body and a plurality of barriers extending vertically upwards from the body, the body defining the plurality of openings. The plurality of openings may be arranged in a plurality of rows along the body, and the each of the plurality of barriers may extend adjacent to at least one row of the plurality of rows of openings. At least one first barrier of the plurality of barriers may extend along an edge of the body and extends a first distance away from the body and at least one second barrier extends along an interior of the body and extends a second distance away from the body, and the second distance may be longer than the first distance.

In some embodiments, the dividing wall arrangement may include a plurality of longitudinal walls and a plurality of lateral walls intersecting the plurality of longitudinal walls to define the plurality of spaces therebetween, and the cover may be configured to rest on a top surface of the dividing wall arrangement in the container. An internal bottom surface of the container may be configured to support a proximal end of the plurality of syringes, and at least a portion of the internal bottom surface may be slanted at an angle relative to an outer bottom surface of the container such that the proximal end of at least one of the plurality of syringes is at a different elevation of at least another one of the plurality of syringes. The internal bottom surface may include a first slanted portion extending in a first direction and a second slanted portion extending in a second direction different from the first direction. The first slanted portion may extend from a first end of the container, and the second slanted portion extends from a second end of the container opposite the first end. The first slanted portion and the second slanted portion may define a peak.

In some embodiments, a first portion of the plurality of spaces may be configured to receive an individual syringe of the plurality of syringes therein, and a second portion of the plurality of spaces may be configured to be not receive an individual syringe therein. The second portion of the plurality of spaces may be arranged to surround the first portion of the plurality of spaces. The second portion of the plurality of spaces may have a volume that is less than a volume of the first portion of the plurality of spaces. At least a portion of each dividing wall of the dividing wall arrangement may be formed from a material selected from the group consisting of card stock, cartonboard, cardboard, SBS paperboard, corrugated cardboard, a polymeric material, a biodegradable polymeric material, recycled pulp-based materials, and combinations of any thereof.

In some embodiments, a packaging system for the sterilization of syringes may include: a plurality of syringes, wherein each syringe of the plurality of syringes may include: a body defining a proximal end and a distal end; a plunger disposed within the body proximate the proximal end; and a tip at the distal end; a dividing wall arrangement defining a plurality of spaces, at least a portion of the plurality of spaces being configured to receive one of the plurality of syringes therein. Each of the plurality of syringes may be received in an upright, substantially parallel configuration and the dividing wall arrangement may divide each individual syringe from one or more adjacent syringes. The packaging system further may include a cover configured to receive the distal ends of the plurality of syringes therethrough; and a container configured to receive the plurality of syringes, the dividing wall arrangement, and the cover therein. The plurality of syringes, dividing wall arrangement, and cover may be received within the container such that each of the plurality of syringes are configured to be sterilized within the container by an outside source.

In some embodiments, an internal bottom surface of the container may be configured to support the proximal end of the syringes, and at least a portion of the internal bottom surface may be slanted such that the proximal end of at least one of the plurality of syringes is at a different elevation of at least another one of the plurality of syringes. A plunger of the at least one of the plurality of syringes may be at least partially offset in a lateral direction from a plunger of the at least another one of the plurality of syringes. The internal bottom surface may include a first slanted portion extending in a first direction and a second slanted portion extending in a second direction different from the first direction, and the first slanted portion may abut the second slanted portion at respective ends thereof.

In some embodiments, the cover may include a body defining a plurality of openings, each of the plurality of openings may be configured to receive the distal end of a syringe of the plurality of syringes therethrough. Each of the plurality of syringes may further include a clip feature connected to the distal end, and each of the plurality of openings may be further configured to receive the clip feature and the distal end of the plurality of syringes therethrough. The cover may further include at least one barrier extending vertically upward from the body, the at least one barrier separating at least a first portion of the plurality of openings from at least a second portion of the plurality of openings. The at least one barrier may include a plurality of barriers, the plurality of barriers extending along a length of the body and separating the plurality of openings into rows.

In some embodiments, a method for sterilizing a plurality of syringes contained in a packaging system may include packaging the plurality of syringes in a syringe packaging system, and irradiating at least a portion of the syringe packaging system with a sterilizing irradiation beam oriented in a direction substantially perpendicular to the longitudinal axis of the plurality of syringes. The syringe packaging system may include a dividing wall arrangement defining a plurality of spaces; a container configured to receive the dividing wall arrangement and a plurality of syringes therein; and an internal bottom surface of the container configured to support a proximal end of the plurality of syringes. At least a portion of the plurality of spaces may be configured to receive an individual syringe of the plurality of syringes therein. The plurality of syringes may be received in an upright, substantially parallel configuration and the dividing wall arrangement divides each individual syringe from one or more adjacent syringes. At least a portion of the internal bottom surface may be slanted at an angle relative to an outer bottom surface of the container such that the proximal end of at least one of the plurality of syringes is at a different elevation of at least another one of the plurality of syringes. A plunger of the at least one of the plurality of syringes may be at least partially offset in a lateral direction from a plunger of the at least another one of the plurality of syringes.

In some embodiments, the sterilizing irradiation beam may include an electron-beam sterilizing irradiation beam. A surface of the plunger of the at least one of the plurality of syringes may receive an irradiation amount of the sterilizing irradiation beam that is substantially similar to an irradiation among of the sterilizing irradiation beam received by a surface of the at least another one of the plurality of syringes.

In some embodiments or aspects, the present disclosure may be characterized by one or more of the following numbered clauses:
Clause 1. A syringe packaging system comprising: a dividing wall arrangement defining a plurality of spaces; and a container configured to receive the dividing wall arrangement and a plurality of syringes therein, wherein at least a portion of the plurality of spaces are configured to receive an individual syringe of the plurality of syringes therein, wherein the plurality of syringes are received in an upright, substantially parallel configuration and the dividing wall arrangement divides each individual syringe from one or more adjacent syringes.
Clause 2. The syringe packaging system of clause 1, further comprising a cover defining a plurality of openings, wherein each opening of the plurality of openings is configured to receive a distal end of a syringe of the plurality of syringes therethrough.
Clause 3. The syringe packaging system of clause 2, wherein each of the plurality of openings comprise at least one slit circumferentially arranged around a periphery of each of the plurality of openings.
Clause 4. The syringe packaging system of clause 3, wherein the each of the plurality of syringes comprise a clip feature located on the distal end, and wherein each of the plurality of openings are configured to receive the clip feature therethrough.
Clause 5. The syringe packaging system of any one of clauses 2 to 4, wherein the cover comprises a body and a plurality of barriers extending vertically upwards from the body, the body defining the plurality of openings, wherein the plurality of openings are arranged in a plurality of rows along the body, and wherein the each of the plurality of barriers extend adjacent to at least one row of the plurality of rows of openings.
Clause 6. The syringe packaging system of clause 5, wherein at least one first barrier of the plurality of barriers extends along an edge of the body and extends a first distance away from the body and at least one second barrier extends along an interior of the body and extends a second distance away from the body, and wherein the second distance is longer than the first distance.
Clause 7. The syringe packaging system of any one of clauses 2 to 6, wherein the dividing wall arrangement comprises a plurality of longitudinal walls and a plurality of lateral walls intersecting the plurality of longitudinal walls to define the plurality of spaces therebetween, and wherein the cover is configured to rest on a top surface of the dividing wall arrangement in the container.
Clause 8. The syringe packaging system of any one of clauses 1 to 7, wherein an internal bottom surface of the container is configured to support a proximal end of the plurality of syringes, and wherein at least a portion of the internal bottom surface is slanted at an angle relative to an outer bottom surface of the container such that the proximal end of at least one of the plurality of syringes is at a different elevation of at least another one of the plurality of syringes.
Clause 9. The syringe packaging system of clause 8, wherein a plunger of the at least one of the plurality of syringes is at least partially offset in a lateral direction from a plunger of the at least another one of the plurality of syringes.
Clause 10. The syringe packaging system of clause 8 or 9, wherein the internal bottom surface comprises a first slanted portion extending in a first direction and a second slanted portion extending in a second direction different from the first direction.
Clause 11. The syringe packaging system of clause 10, wherein the first slanted portion extends from a first end of the container and the second slanted portion extends from a second end of the container opposite the first end.
Clause 12. The syringe packaging system of clause 11, wherein the first slanted portion and the second slanted portion define a peak.
Clause 13. The syringe packaging system of any one of clauses 1 to 12, wherein a first portion of the plurality of spaces are configured to receive an individual syringe of the plurality of syringes therein and a second portion of the plurality of spaces are configured to be not receive an individual syringe therein.
Clause 14. The syringe packaging system of clause 13, wherein the second portion of the plurality of spaces are arranged to surround the first portion of the plurality of spaces.
Clause 15. The syringe packaging system of clause 14, wherein the second portion of the plurality of spaces has a volume that is less than a volume of the first portion of the plurality of spaces.
Clause 16. The syringe packaging system of any one of clauses 1 to 15, wherein at least a portion of each dividing wall of the dividing wall arrangement is formed from a material selected from the group consisting of card stock, cartonboard, cardboard, SBS paperboard, corrugated cardboard, a polymeric material, a biodegradable polymeric material, recycled pulp-based materials, and combinations of any thereof.
Clause 17. A packaging system for the sterilization of syringes, the system comprising: a plurality of syringes, wherein each syringe of the plurality of syringe comprises: a body defining a proximal end and a distal end; a plunger disposed within the body proximate the proximal end; and a tip at the distal end; a dividing wall arrangement defining a plurality of spaces, at least a portion of the plurality of spaces being configured to receive one of the plurality of syringes therein, wherein each of the plurality of syringes are received in an upright, substantially parallel configuration and the dividing wall arrangement divides each individual syringe from one or more adjacent syringes; a cover configured to receive the distal ends of the plurality of syringes therethrough; and a container configured to receive the plurality of syringes, the dividing wall arrangement, and the cover therein, wherein the plurality of syringes, dividing wall arrangement, and cover are received within the container such that each of the plurality of syringes are configured to be sterilized within the container by an outside source.
Clause 18. The packaging system of clause 17, wherein an internal bottom surface of the container is configured to support the proximal end of the syringes, and wherein at least a portion of the internal bottom surface is slanted such that the proximal end of at least one of the plurality of syringes is at a different elevation of at least another one of the plurality of syringes.
Clause 19. The syringe packaging system of clause 18, wherein a plunger of the at least one of the plurality of syringes is at least partially offset in a lateral direction from a plunger of the at least another one of the plurality of syringes.
Clause 20. The packaging system of clause 18 or 19, wherein the internal bottom surface comprises a first slanted portion extending in a first direction and a second slanted portion extending in a second direction different from the first direction, and wherein the first slanted portion abuts the second slanted portion at respective ends thereof.
Clause 21. The packaging system of any one of clauses 17 to 20, wherein the cover comprises a body defining a plurality of openings, wherein each of the plurality of openings is configured to receive the distal end of a syringe of the plurality of syringes therethrough.
Clause 22. The packaging system of clause 21, wherein each of the plurality of syringes further comprise a clip feature connected to the distal end, and wherein each of the plurality of openings are further configured to receive the clip feature therethrough.
Clause 23. The packaging system of clause 21 or 22, wherein the cover further comprises at least one barrier extending vertically upward from the body, the at least one barrier separating at least a first portion of the plurality of openings from at least a second portion of the plurality of openings.
Clause 24. The packaging system of clause 23, wherein the at least one barrier comprises a plurality of barriers, the plurality of barriers extending along a length of the body and separating the plurality of openings into rows.
Clause 25. A method for sterilizing a plurality of syringes contained in a packaging system, the method comprising: packaging the plurality of syringes in a syringe packaging system, the syringe packaging system comprising: a dividing wall arrangement defining a plurality of spaces; a container configured to receive the dividing wall arrangement and a plurality of syringes therein; and an internal bottom surface of the container configured to support a proximal end of the plurality of syringes, wherein at least a portion of the plurality of spaces are configured to receive an individual syringe of the plurality of syringes therein, wherein the plurality of syringes are received in an upright, substantially parallel configuration and the dividing wall arrangement divides each individual syringe from one or more adjacent syringes, wherein at least a portion of the internal bottom surface is slanted at an angle relative to an outer bottom surface of the container such that the proximal end of at least one of the plurality of syringes is at a different elevation of at least another one of the plurality of syringes, and wherein a plunger of the at least one of the plurality of syringes is at least partially offset in a lateral direction from a plunger of the at least another one of the plurality of syringes; and irradiating at least a portion of the syringe packaging system with a sterilizing irradiation beam oriented in a direction substantially perpendicular to the longitudinal axis of the plurality of syringes.
Clause 26. The method of clause 25, wherein the sterilizing irradiation beam comprises an electron-beam sterilizing irradiation beam.
Clause 27. The method of clause 25 or 26, wherein a surface of the plunger of the at least one of the plurality of syringes receives an irradiation amount of the sterilizing irradiation beam that is substantially similar to an irradiation amount of the sterilizing irradiation beam received by a surface of the at least another one of the plurality of syringes.

### BRIEF DESCRIPTION OF THE DRAWING(S)

FIG. 1 is a perspective view of a syringe packaging system according to one embodiment of the present disclosure;

FIG. 2 is a partially exploded view of the syringe packaging system of FIG. 1;

FIG. 3 is a perspective view of a cover and dividing wall arrangement used to separate syringes in the syringe packaging system of FIG. 1;

FIG. 4 is a top view of cover, dividing wall arrangement, and syringes of FIG. 3;

FIG. 5 is an exploded view of the cover, dividing wall arrangement and syringes of FIG. 3;

FIG. 6 is a perspective view of the cover and dividing wall arrangement of the syringe packaging system of FIG. 1;

FIG. 7 is a perspective view of a cover of the syringe packaging system of FIG. 1;

FIG. 8 is a top view of the cover of FIG. 7 with unfolded end barriers;

FIG. 9 is a top view of the cover of FIG. 7 with folded end barriers;

FIG. 10 is a side view of the cover of FIG. 7;

FIG. 11 is a perspective view of multiple syringe packaging systems of FIG. 1 packaged together in a bulk packaging configuration;

FIG. 12 is a partially-transparent perspective view of a container of a syringe packaging system having a slanted internal bottom surface according to another embodiment of the present disclosure;

FIG. 13 is a partially-transparent perspective view of a syringe packaging system including a plurality of syringes and cover with the container of FIG. 12;

FIG. 14 is a side view of the container of FIG. 12 in an unassembled arrangement;

FIG. 15 is a partially-transparent side view of the syringe packaging system of FIG. 13 showing direction of e-beam irradiation;

FIG. 16 is a partially transparent side view of a syringe packaging system showing direction of e-beam irradiation and having a container according to another embodiment of the present disclosure;

FIG. 17 is a perspective outside view of a container of a syringe packaging system according to another embodiment of the present disclosure;

FIG. 18 is a partially transparent perspective view of a syringe packaging system using the container of FIG. 17;

FIG. 19 is an exploded view of the syringe packaging system of FIG. 18;

FIG. 20 is a perspective view of a syringe packaging system showing the container and dividing wall arrangement according to another embodiment of the present disclosure;

FIG. 21 is a perspective view of another packaging system for a plurality of individually packaged stored medical devices according to another embodiment of the present disclosure;

FIG. 22 is a partial perspective view of the packaging system of FIG. 21, showing the removal process of one of stored medical devices from the plurality of stored medical devices;

FIG. 23 is a perspective view of the packaging of a plurality of stored medical devices of the packaging system of FIG. 21;

FIG. 24 is a partially transparent side view of the packaging system of FIG. 21;

FIG. 25A is a perspective view of a syringe packaging according to another embodiment of the present disclosure;

FIG. 25B is a second perspective view of the syringe packaging of FIG. 25A;

FIG. 26 is a perspective view of a packaging system for disposable tubing components according to another embodiment of the present disclosure;

FIG. 27 is a partially transparent side view of the packaging system of FIG. 26 according to one embodiment of the present disclosure;

FIG. 28 is a second partially transparent side view of the packaging system of FIG. 26 according to one embodiment of the present disclosure;

FIG. 29 is a perspective view of a packaging system for disposable tubing components according to another embodiment of the present disclosure;

FIG. 30A is a perspective view of a packaging according to another embodiment of the present disclosure;

FIG. 30B is a second perspective view of the packaging of FIG. 30A;

FIG. 31 is a partially transparent perspective view of a packaging for a disposable fluid path component according to another embodiment of the present disclosure; and

FIG. 32 is a partially transparent perspective view of the packaging of FIG. 31.

### DETAILED DESCRIPTION

As used herein, the singular form of “a”, “an”, and “the” include plural referents unless the context clearly dictates otherwise. The term “includes” is synonymous with “comprises”. The term “at least” is synonymous with “greater than or equal to”.

As used herein, the terms “parallel” or “substantially parallel” mean a relative angle as between two objects (if extended to theoretical intersection), such as elongated objects and including reference lines, that is from 0° to 5°, or from 0° to 3°, or from 0° to 2°, or from 0° to 1°, or from 0° to 0.5°, or from 0° to 0.25°, or from 0° to 0.1°, inclusive of the recited values.

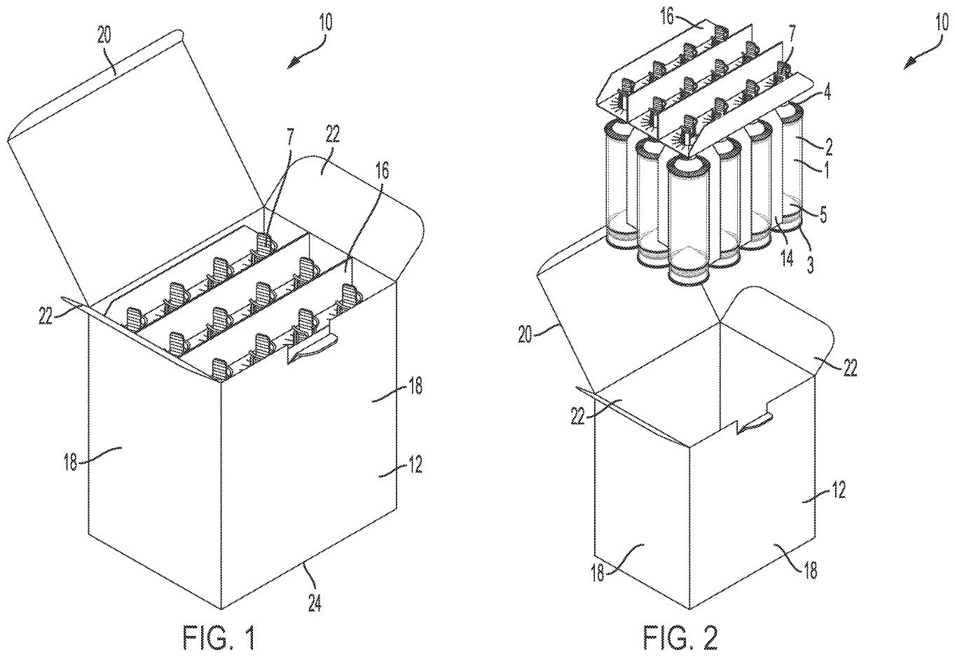

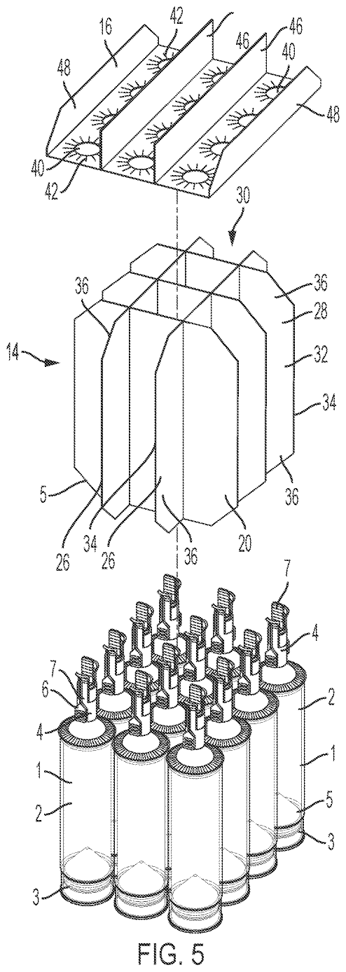

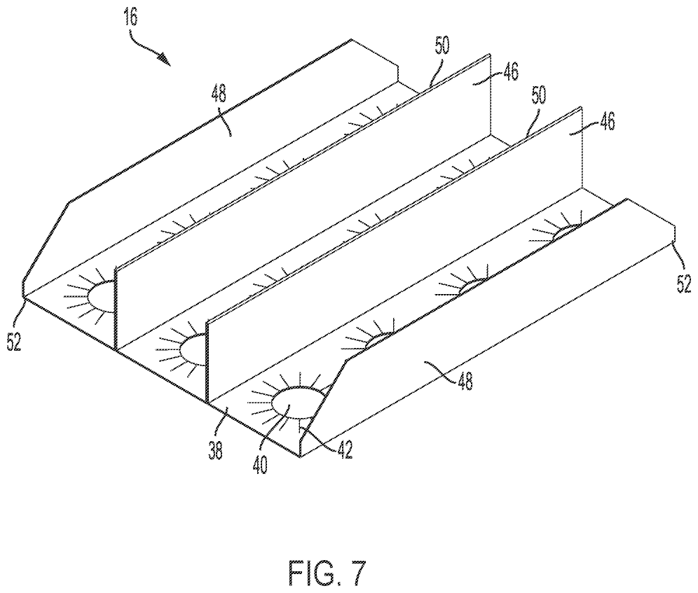

FIGS. 1-10 show a syringe packaging system 10 according to one embodiment of the present disclosure. The packaging system 10 is arranged to support a plurality of syringes 1 to be packaged, sterilized, and shipped without damaging the plurality of syringes 1 or their fluid pathways. The system 10 includes eco-friendly packaging that protects the syringes 1 while minimizing the amount of material used and incorporating recyclable materials into the manufacturing process of the packaging system 10. The materials that are used are, when possible, are environmentally friendly materials, meaning that may include post-consumer content, and/or can be recycled or reused. When the syringes 1 are removed from system 10, the waste produced by the leftover parts of system 10 will be minimized and/or readily recycled.

An example of a syringe 1 that can be used in such a packaging system 10 includes an angiography syringe that is disclosed in International Patent Application Publication WO 2022/035791, the disclosure of which is hereby incorporated by reference in its entirety. Other medical devices and other configurations of syringes may also be used in this system 10 and one will understand how the system 10 may be adjust to account for different devices. However, a syringe 1 will be discussed in connection with the discussion of packaging system 10 in this disclosure. For reference, each syringe 1 of the plurality of syringes includes a body 2 having a proximal end 3 and a distal end 4. In certain embodiments, the plunger 5 may be disposed at the proximal end 3 of the syringe, although it is understood that the plunger 5 may be located longitudinally anywhere in the body 2, and a nozzle 6 is located at the distal end 4, wherein the nozzle 6 is configured for connection with one or more fluid path components. Fluid or other medicament may be disposed in the body between the plunder 5 and distal ends 4, for example, if the syringe is a pre-filled configuration, or after filling the syringe with the fluid or medicament. In other embodiments, the syringes 1 may be empty, i.e., contain no liquid, and be filled after the transport and receipt of the packaging system 10 by an end user. The syringes 1 may also have a clip 7 or connector member disposed within the nozzle 6. Examples of this are described in International Patent Application Publication No. WO 2021/168076, the disclosure of which is hereby incorporated by reference in its entirety. By using the packaging system 10 described herein, the syringes 1 may be sterilized and transported the end user, such as a hospital.

According to various embodiments, the plurality of syringes 1 may be packaged with a “sterile fluid path” configuration. As used herein, the term “sterile fluid path” means that the interior portions of the syringes 1 and other medical devices, i.e., the portions of the medical devices that come into contact with the fluid in the fluid path, are substantially isolated from the atmospheric environment, for example by sealing the nozzle 6 with a cap or other sealing member so that the interior contents of the syringe 1 may be isolated from any potential microbial contaminants during shipping and injector set-up. Once the interior portions are substantially isolated, the syringe 1 or medical device component may be subjected to a sterilization process, such as by exposure to a sterilizing compound, such as ethylene oxide gas, or by irradiation with a sterilization beam, such as an electron beam (commonly referred to as an “e-beam”) to sterilize the surfaces and interior volumes of the syringe 1 or medical device component. The sterilized syringe 1 or medical device component may be shipped and handled without worry that the interior fluid path will be contacted by any microbial contaminants and the sterilization of the surfaces and interior volumes will be maintained. The sterile fluid path configuration requires only the interior fluid path surfaces and volumes be maintained in a sterilized environment while other, exterior portions of the syringe 1 or medical device component need not be maintained in a sterilized environment. Such a configuration may reduce costs as less surface area is required to maintain a sterilized environment, while still ensuring patient safety and reduced exposure to microbial contamination.

Referring first to FIGS. 1 and 2, according to one embodiment, the packaging system may include a bulk container 12, such as a box, to hold the plurality of syringes 1 and other parts of the system 10 therein. According to various embodiments, the plurality of syringes 1 may be held in an upright, substantially parallel configuration. The system 10 also includes a dividing wall arrangement 14 to separate the syringes 1 and a cover 16 to extend over the syringes 1, the features of which will be discussed in greater detail below. The container 12 is shown as a box that may be made from materials such as cardboard, card stock, cartonboard, SBS paperboard, corrugated cardboard, a polymeric material, a biodegradable polymeric material, recycled pulp-based materials, and combinations of any thereof, or other suitable materials to support and secure the syringes 1 when placed therein. In some embodiments, the container 12 or portions thereof may be made from biodegradable, recyclable, or post-consumer/recycled products to enhance the eco-friendly nature of the packaging system 10. The container 12 includes sidewalls 18 and defines a lid 20 and flaps 22 for opening the container 12 and accessing the syringes 1 via the top of the container 12. The container 12 also includes a bottom wall 24 that may support the proximal ends 3 of syringes 1 inside the container 12.

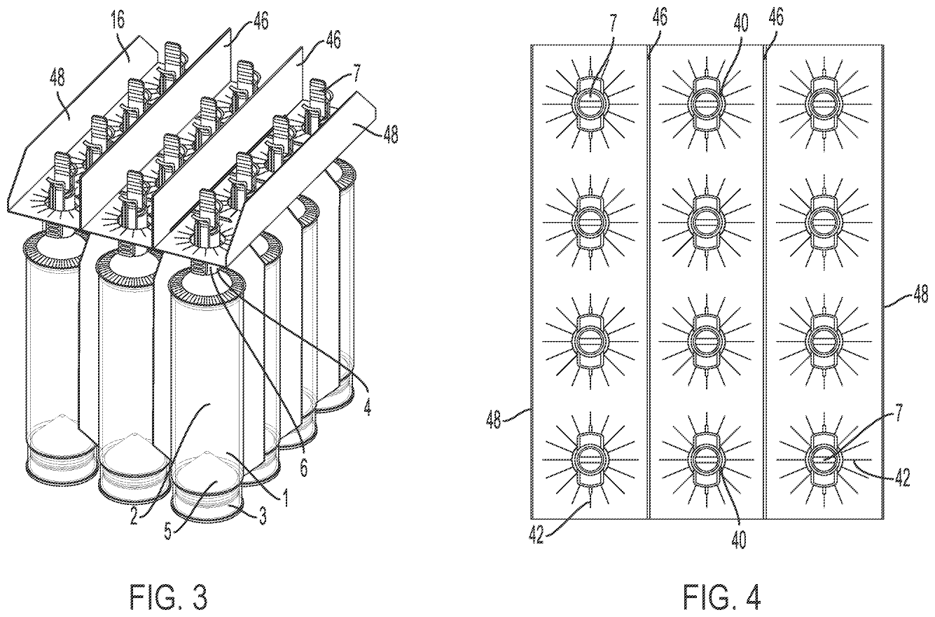

Referring to FIGS. 2-6, the dividing wall arrangement 14 is shown to prevent the syringes 1 from contacting adjacent syringes 1 during packaging, shipping, and storage. Such a dividing wall arrangement 14 may prevent surface scratches on the syringe exterior, damaging, or breaking of various syringe features during shipping and storage. The dividing wall arrangement 14 is arranged to form a grid-like or matrix-like structure that defines a plurality of receiving spaces 30 to hold at least a portion of the syringes 1 therein. The dividing wall arrangement 14 includes longitudinal walls 26 and lateral walls 28 that define the receiving spaces 30 therebetween for receiving at least a portion of the body 2 of syringe 1. As shown, the longitudinal walls 26 run substantially parallel to one another and the lateral walls run substantially parallel 28 to one another. The walls 26, 28 may be arranged to intersect and define 90 degree angles at the intersections. These intersections define the receiving spaces 30 for the syringes 1. The longitudinal walls 26 are arranged relative to the lateral walls 28 to isolate one syringe 1 from the others within a single receiving space 30. Although the longitudinal walls 26 are shown as intersecting the lateral walls 28 at 90° angles, other arrangements may be considered, so long as the intersections of the various walls define spaces adequate to hold and isolate a syringe 1 or another medical device. The dividing wall arrangement 14 may be manufactured from cardboard, card stock, or any other suitable textile product as described herein. In some embodiments, at least a portion of the dividing wall arrangement 14 may be made from recyclable or recycled/post-consumer materials to enhance the eco-friendly nature of the packaging system 10. The dividing wall arrangement 14 may also be collapsible, meaning that it may fold flat at or near the intersections of the longitudinal walls 26 and lateral walls 28. This allows the dividing wall arrangement 14 to be folded over on itself in order to take up a smaller amount of space when not in use inside a container 12, such as when placed in a waste or recycling container. In some embodiments, the dividing wall arrangement 14 may be reusable and/or recyclable. In other embodiments, the walls 26, 28 may have corresponding slots and pegs to allow for assembly in the manner shown in the figures and disassembly into the individual wall components for ready recycling or disposal. As shown in FIGS. 3, 5, and 6, in certain embodiments the longitudinal walls 26 and/or the lateral walls 28 have end portions 32 that are tapered, although in other components the longitudinal walls 26 and/or lateral walls 28 may have perpendicular corners. The end portions 32 may have a straight edge portion 34 and two angled edge portions 36 defining the taper. The exact shape and size of the taper may be determined by the size of the syringe 1 or medical device that is held within the dividing wall arrangement 14, so that each individual syringe 1 can remain isolated from the other syringes 1 in the system. The longitudinal walls 26 and lateral walls 28 are sized so that, during transport, any movement or jostling of the containers 12 is minimized and only results in an individual syringe 1 contacting an adjacent wall 26, 28 instead of another syringe 1, thus preventing adjacent syringes from contacting one another and potentially scratching or otherwise marring a surface of one or both syringes. In various embodiments, the ends of the dividing wall arrangement 14 are open, meaning a syringe 1 located along one of these ends has its receiving space 30 further defined by one of the sidewalls 18 of container 12.

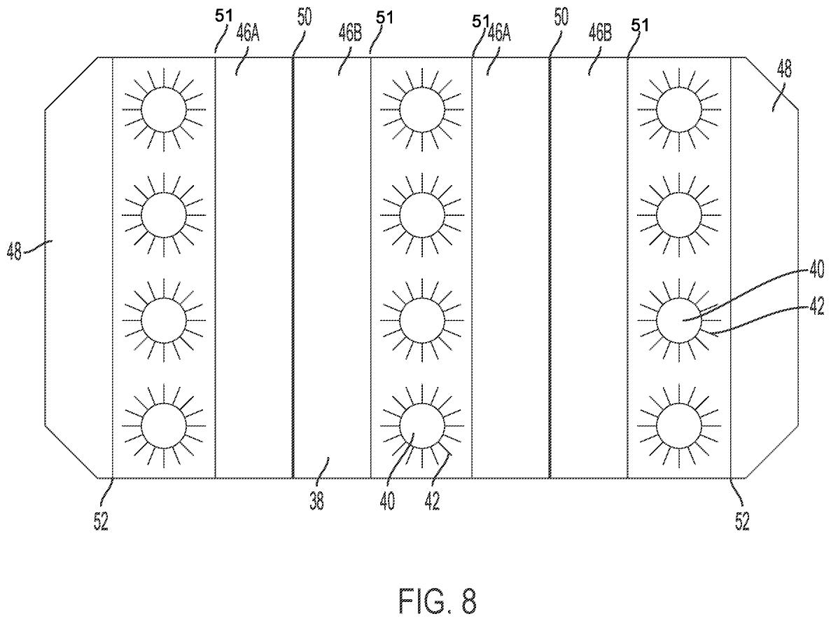

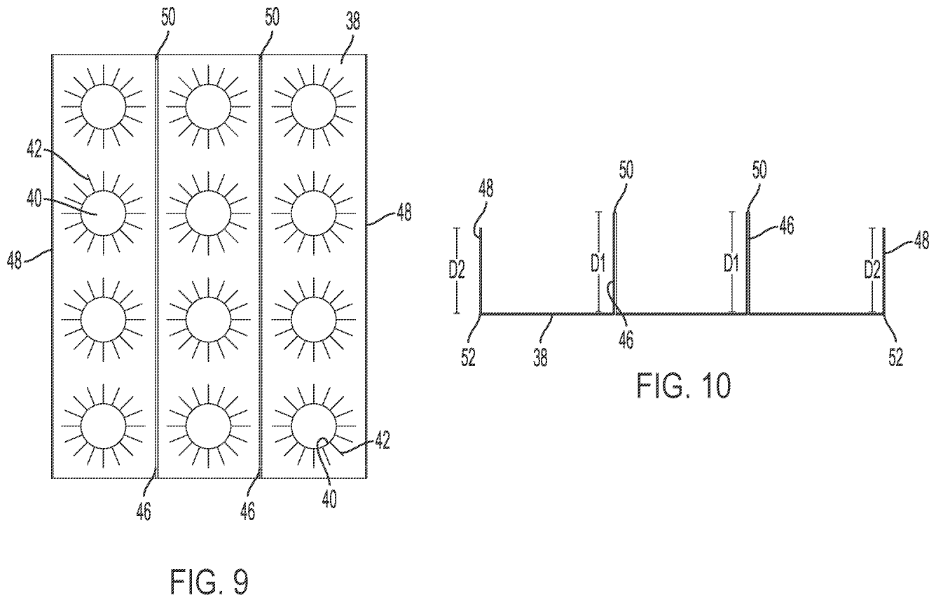

Referring to FIGS. 2-10, an embodiment of cover 16 is shown. The cover 16 is arranged to be placed on top of the dividing wall arrangement 14 to secure the distal ends 4 of the syringes 1 and minimize both longitudinal and lateral movement of the syringes during shipping and handling of the packaging system. By securing the distal ends 4 of the syringes 1 with the cover 16, the syringes 1 can be held upright and substantially immobile within the container 12. The cover 16 has a body 38 that defines a plurality of openings 40 for receiving at least a portion of the distal ends 4 of the syringes 1 therethrough. The openings 40 may secure the distal ends 4 via a friction fit. In certain embodiments, the shape of each of the openings 40 may be such that the nozzle 6 may open the opening 40 when the cover 16 is placed into the packaging system 10 and the syringe 1 is inserted. In various embodiments, the opening 40 may be secured around a narrower part of the nozzle 6 or distal end 4 after the end of the nozzle 6 has passed through the opening to secure the syringe 1. In certain embodiments, the openings 40 may include one or more slits 42 extending radially outward relative to the periphery of the opening 40, for example, to provide some give when the distal end 4 is pushed through the opening 40. Such an arrangement may accommodate the clips 7, which may be wider than the nozzle 6. For example, the slits 42 may allow for insertion of at least a portion of the flexible legs or other features associated with the clips 7. In the figures, only some of the openings 40 and slits 42 are labeled for clarity. As shown in FIGS. 8 and 9, a portion of the plurality of openings 40 may be arranged in rows along the body 38 of the cover, and this arrangement results in the syringes 1 being arranged in rows within the container 12, for example when the syringes 1 are maintained within the dividing wall arrangement 14. In other embodiments, different arrangements may be used for storing the syringes 1 within the container 12. For example, the openings 40 may be arranged in a circular, square, rectangular, or oval arrangement. In instances such as this, the shape and arrangement of the dividing wall arrangement 14 may also be changed to accommodate the syringes 1 and cover 16.

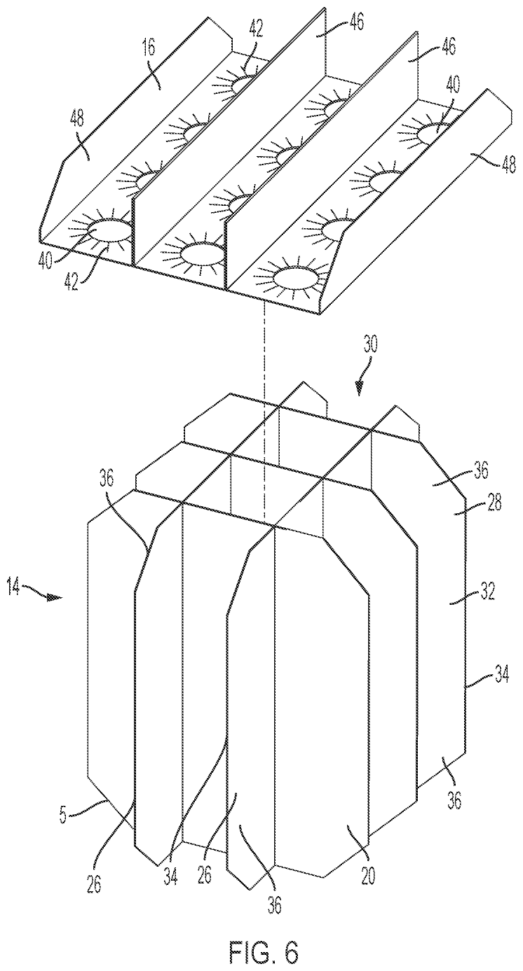

According to various embodiments, the cover 16 may also include one or more barriers 44 that separate the rows of openings from one another and to locate cover 16 within the packaging system 10 to hold the cover in place relative to the packaging system 10. As shown, the cover 16 may include a plurality of internal barriers 46, such as the two internal barriers 46 illustrated in FIGS. 6, 7, and 10 and two end barriers 48, with each of the barriers 46, 48 extending parallel to one another. The internal barriers 46 extend along an interior portion of the body 38, and the end barriers 48 extend along outer edges of the body 38. The barriers 44 may further serve to isolate the distal ends 4 of syringes 1 from one another. As shown in various embodiments, the internal barriers 46 may extend a first distance D1 away from the body 38, and the end barriers 38 may extend a second distance D2 that is shorter than the first distance D1. The lengths of the distances D1, D2 are such that they extend beyond the distal end 4 of the syringes 1 held within the cover 16. This ensures that the syringes 1 are maintained within the container 12 without significant movement and prevent the distal ends 4 of the syringes 1 from contacting a top surface or lid 20 of the container 12 during shipping and storage. Further, the plurality of internal barriers 46 and the end barriers 48 of cover 16 may provide improved structural integrity to lid 20 of container 12, to increase the weight bearing load and prevent crushing of the top of container 12 during shipping and storage. Any number of internal barriers 46 may be defined depending on the number of syringes 1 being held by cover 16. In other embodiments, the end barriers 48 may be omitted. In these embodiments, sidewalls 18 of container 12 may serve as a de facto end barrier 48 for cover 16.

As illustrated in FIG. 8, in some embodiments, the body 38 of cover 16 may include different folds 50, 52 to form the barriers 46, 48. Internal folds 50 and 51may be provided along internal lengths of the body 38. The internal folds define a first side 46A and a second side 46B of an internal barrier 46. By folding the body 38 at the internal folds 50 and 51, the first aide 46A contacts the second side 46B to form the internal barrier 46. End folds 52 may be provided a distance away from an outer edge of the body 38, so that the end barriers 48 may be folded into place at the end of the body 38. In other embodiments, the cover 16 is formed with the barriers 46, 48 extending from the body 38 without needing to fold any part of the body 38.

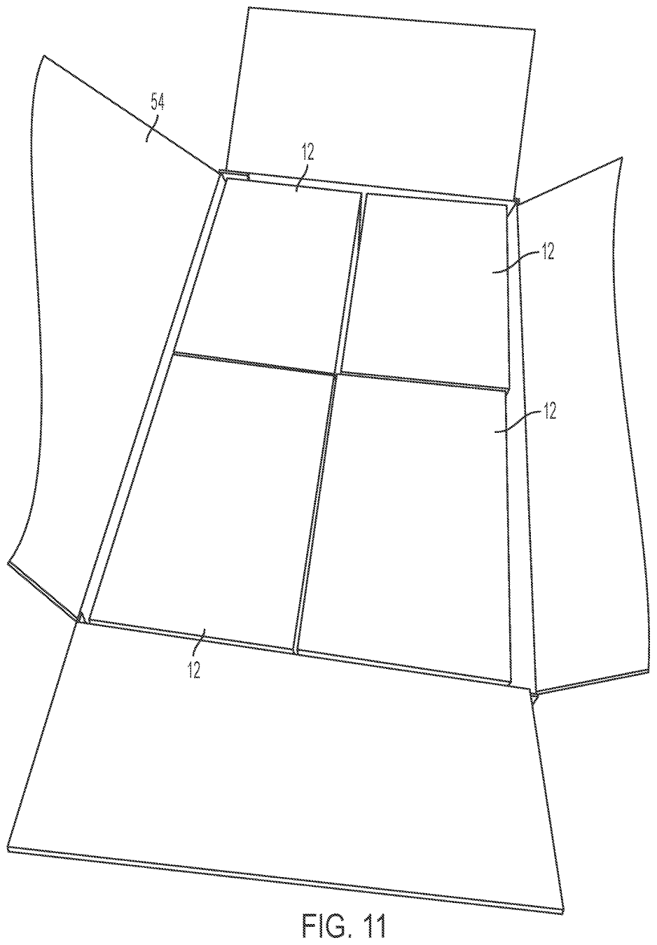

Referring to FIGS. 1-10, the packaging system 10 may be assembled by inserting the dividing wall arrangement 14 into the container 12. Then, the syringes 1 may then be individually inserted into the receiving spaces 30 formed between the longitudinal walls 26 and lateral walls 28 of the dividing wall arrangement 14. Then, the cover 16 may be folded and the folded cover 16 is placed on top of the dividing wall arrangement 14, which forces the distal ends 4 and/or clips 7 of the syringes 1 through one of the openings 40 formed in the body 38 of the cover 16. Then, the flaps 22 and lid 20 of the container 12 are closed. The barriers 46, 48 are sized so that they do not interfere with the closing of the flaps 22 and lid 20 and abut the interior surface of the lid 20 when the container 12 is in the closed configuration. The container 12 may be further secured by sealing the lid 20 with tape, glue, staples, or other known methods. With reference to FIG. 11, the individual sealed containers 12 may be placed in a larger, bulk shipping container 54 with other containers 12 for sterilization and/or shipping. The bulk shipping container 54 may be loaded onto a pallet with other bulk shipping containers or shipped individually. The bulk shipping containers 54 may be shrink wrapped or packaged further to facilitate bulk shipping. According to various embodiments, the plurality of syringes may be sterilized using a sterilization method, such as exposure to ethylene oxide or e-beam irradiation, either once packaged and sealed in container 12 or once a plurality of containers 12 are packaged into bulk shipping container 54, either alone or palletized. Using such a sterilization process, interior surfaces of the plurality of syringes may be sterilized as necessary for a sterile fluid path packaging configuration.

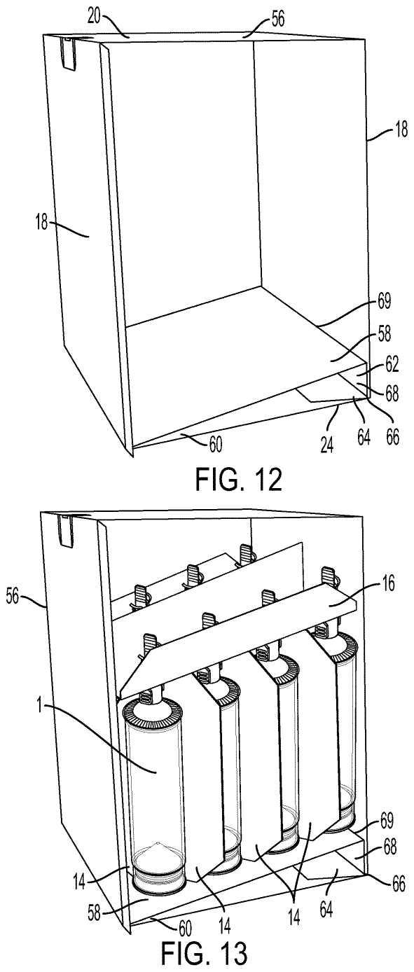

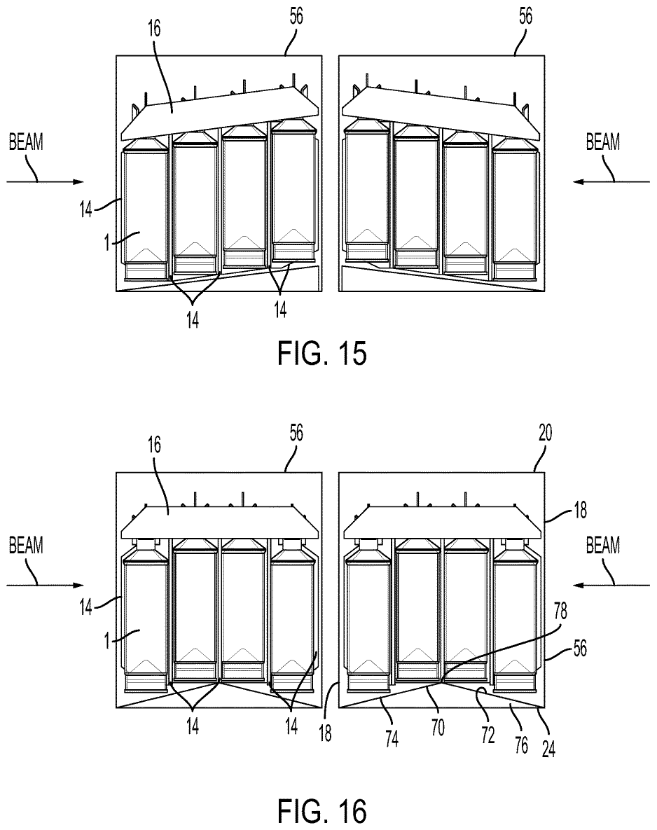

Referring now to FIGS. 12-16, another container 56 is shown according to another embodiment of the present disclosure. A plurality of syringes 1 may be held inside the containers 12, 56 discussed in this disclosure and may be sterilized via certain processes, such as electron beam (“e-beam”) irradiation or exposure to a sterilization agent, such as ethylene oxide. As shown in FIGS. 15 and 16, the container 12 may be irradiated with e-beam irradiation from a side and passes through the container sidewalls 18, syringes 1, dividing wall arrangement 14 and cover 16 as necessary as the e-beam passes through these features, see e.g., FIGS. 15 and 16. All of the elements held within the containers 12, 56 may all undergo this sterilization process and the interior portions of the sterile fluid paths of the plurality of syringes may be effectively sterilized. According to various embodiments, the packaging configuration of the plurality of syringes 1 may impact the effectiveness and consistency of the sterilization process. For example, in some instances, the sterilization process may be impacted by the thickness and overlap of the plungers 5 within the syringe bodies 2, meaning the presence of a plunger 5 may impact the strength of and exposure to the electron beam irradiation as that beam passes through and interacts with the material associated with the one or more plungers 5. That is, in conventional packaging arrangements where the syringes are packaged side by side in a substantially parallel arrangement, the plungers 5 within the plurality of syringes 1 may be arranged at the same lateral portion of the packaging configuration. When irradiated with e-beam irradiation, the thickness and overlap of the plunger material may reduce the strength of the e-beam irradiation experienced by plungers near the interior of the packaging system relative to the strength of the e-beam irradiation experienced by the plungers near the exterior of the packaging system. As a result, the packaging system may require additional or longer e-beam irradiation times to ensure sufficient sterilization of all plunger surfaces. To address this situation, according to certain embodiments, the container 56 may include a slanted internal bottom surface 58 for supporting the syringes 1, so that the syringes 1 are tiered or staggered within the container 56 and, correspondingly, the plungers are staggered or laterally offset within the container relative to the longitudinal axis of the plurality of syringes. The staggered arrangement of the plurality of syringes 1 inside of the container 56 ensures that the electron beam passes through fewer plungers 5 and less plunger material within the container 56 than if the syringes 1 were secured at the same level. This means that the irradiation process will be more effective as the electron beam is absorbed by and passes through less plunger material during the sterilization process, thus requiring less exposure to the e-beam and ensuring a faster sterilization process.

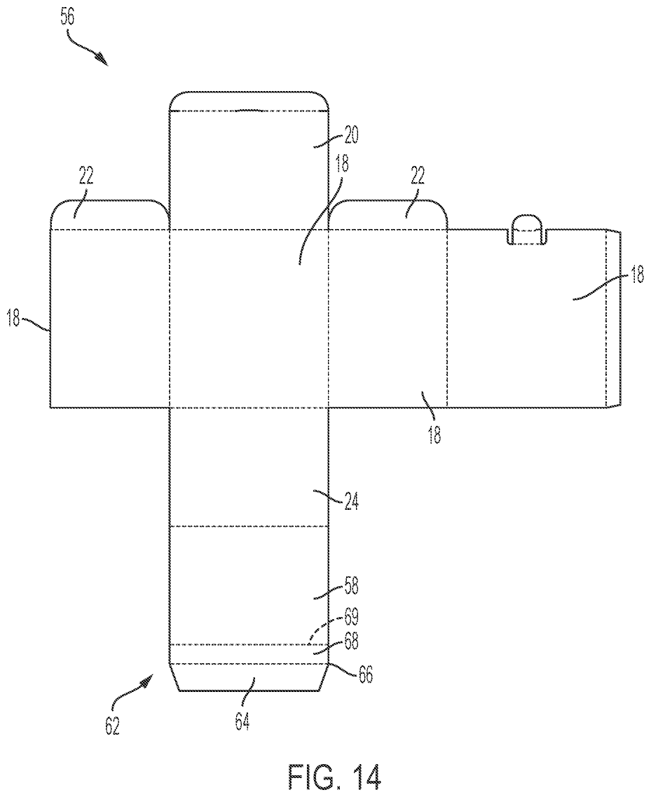

With reference to FIGS. 12-15, according to certain embodiments the container 56 still has sidewalls 18, lid 20, flaps 22, and a bottom surface 24, like the container 12 discussed above in connection with FIGS. 1-11. However, container 56 may also include a slanted internal bottom surface 58 created by a folded over portion 62 as described herein. The folded over portion 62 may be folded over on itself within the container 56 so that an angle 60 is formed between the internal bottom surface 58 and the bottom surface 24 (referred to now as the external bottom surface) of container 56, which creates the slant of the internal bottom surface 58 relative to the longitudinal axis of container 56. The folded over portion 62 includes a first side 64 that contacts an internal surface of the external bottom surface 24, a first fold 66, a second side 68 that contacts a sidewall 18, and a second fold 69 that contacts the same sidewall 18 as the second side 68. The first fold 66 is arranged so that it extends along a corresponding fold defined by the sidewall 18 and the external bottom surface 24 to create the angle that allows the internal bottom surface 58 to be slanted relative to the external bottom surface 24. Other features and methods which result in a staggered upright packaging arrangement of the syringes 1 are also contemplated and are considered within the scope of this packaging embodiment. The assembly of a syringe packaging system utilizing the container 56 and slanted internal bottom surface 58 may be the same as the syringe packaging system 10 discussed above in connection with FIGS. 1-11. That is, the syringe packaging system 10 may include container 56 and one or more of dividing wall arrangement 14 and cover 16. The flexible nature of the materials that form dividing wall arrangement 14 and cover 16 allow for them to function in the same manner as discussed above even with the syringes 1 being staggered along the slanted internal bottom surface 58. FIG. 14 illustrates an embodiment of the container 56 in the unfolded configuration.

With reference to FIG. 16, another embodiment of a staggered syringe packaging system and arrangement is shown. Instead of having a single internal bottom surface 58 that is slanted in one direction as shown in FIG. 13, according to this embodiment, the container 56 includes an internal bottom surface 58 is slanted in two opposing directions. Specifically, the internal bottom surface 58 includes a first slanted portion 70 and a second slanted portion 72. The first slanted portion 70 defines an angle 74 with the bottom surface 24, and the second slanted portion 72 defines an angle 76 with the bottom surface 24. Both slanted portions 70, 72 may meet at a peak 78 along the internal bottom surface 58 allowing for two tiered arrangements of the plurality of syringes 1 along internal bottom surface 58 in a chevron configuration. Irradiation by electron beam sterilization perpendicular to the longitudinal axis of the container in this embodiment allows the beam to irradiate the staggered bodies of the plungers within the staggered syringes for effective and complete irradiation and sterilization. In this embodiment, the syringes 1 may be held within two dividing wall arrangements 14 and covers 16. For example, the syringes 1 that are supported by the first slanted portion 70 may be held within a first dividing wall arrangement 14A, and the syringes that are supported by the second slanted portion 72 may be held within a second dividing wall arrangement 14B. The syringes 1 may also have separate covers 16A, 16B corresponding to the syringes 1 held on one of the slanted portions 70, 72. In other embodiments, as shown in FIG. 16, a single dividing wall arrangement 14 and cover 16 may be bent to account for the differences in the slanted portions 70, 72, so that the single dividing wall arrangement 14 and cover 16 may be used with all of the syringes 1 in the container 56.

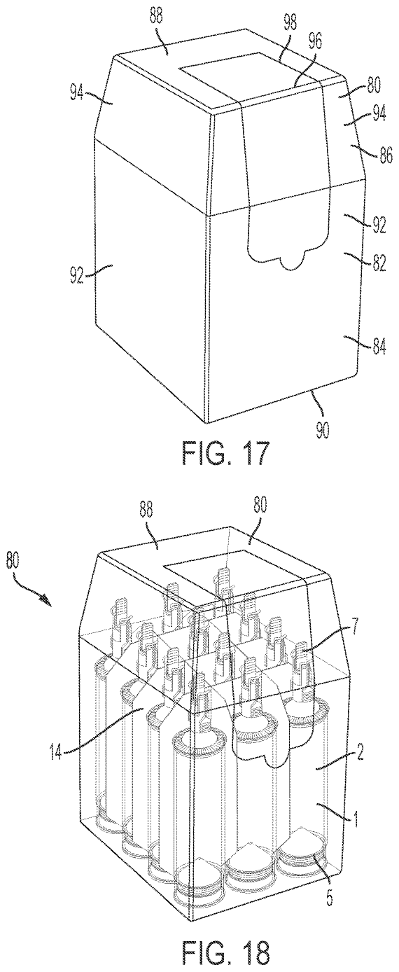

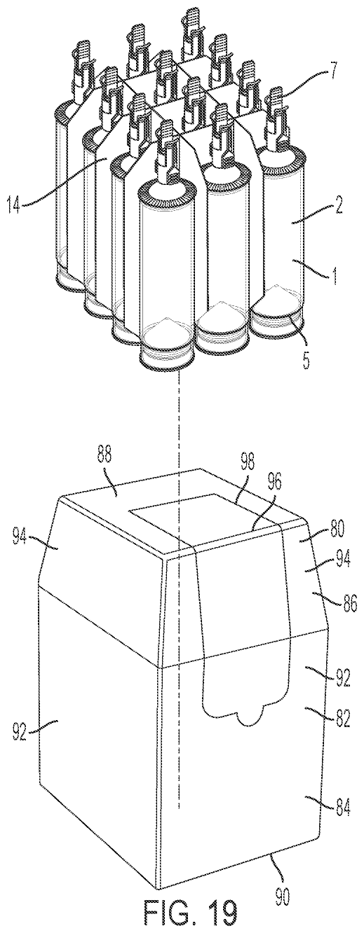

With reference to FIGS. 17, 18, and 19, another embodiment and configuration of a syringe container 80 is shown. According to this embodiment, container 80 has a body 82 with a main body portion 84 and a top body portion 86 extending from the main body portion 84 and including a top surface 88. The main body portion 84 is cube-like and has a bottom surface 90 and sidewalls 92 extending therefrom. The top body portion 84 has sidewalls 94 that may be arranged at an angle relative to the sidewalls 92 of the main body portion 84. This arrangement means the top body portion 86 is partially tapered in the direction extending inward from the main body portion 84. An opening cover 96 may extend from the top surface 88 down over at least a portion of front sidewall 94 of the top body portion 86 and at least a portion front sidewall 92 of the main body portion 84. Perforations 98 may extend along all or part of the opening cover 96 to allow opening cover 96 to be opened or torn away from the container 80 to gain access to the syringes 1 inside of the container 80. As shown in FIG. 18, a plurality of syringes 1 are located inside of the container 80 with a dividing wall arrangement 14, such as the various dividing wall arrangements described herein. The tapered nature of the top body portion 86 may help secure the distal ends of the syringes 1 and prevent movement of the syringes during shipping and handling and means a cover 16 may be omitted due to the interference that may occur between the barriers 46, 48 and the top body portion 86. However, in certain embodiments, a cover 16 may be used as described herein with appropriate modifications to accommodate the shape of the top body portion 86. As shown, the syringes 1 and dividing wall arrangement 14, and cover 16 if present, may be largely located within the main body portion 84 but may at least partially extend into top body portion 86. In other embodiments, the syringes 1 and dividing wall arrangement 14, and cover 16 if present, may be entirely located within the main body portion 84.

In some embodiments, the container 80 may be used as a shelf box packaging and the syringes 1 may include one or more dust caps associated with the medical connector, including a tamper evident or tamper resistant seal to ensure sterility of the syringes 1. Non-limiting examples of the medical connector are shown and described in International Patent Application Publication No. WO 2021/168076. Non-limiting examples of dust caps and tamper evidence features are described and shown in International Patent Application Publication Nos. WO 2023/244580 and WO 2023/212177, the disclosures of which are incorporated herein by this reference. Multiple containers 80 may be packaged in at least one bulk packaging, such as a shipping case. For example, in one embodiment, a plurality of six containers 80 may be packaged into a bulk packaging container for sterilization and shipping, with the interior fluid containing volume of the syringes 1 being sterilized using the sterile fluid path configuration as described herein. In other embodiments, the syringes 1 may be sterilized after being placed into the container 80, for example by e-beam irradiation of the individual container 80, a plurality of containers 80 packaged within a bulk packaging container, or a plurality of bulk packaging containers, each containing a plurality of containers 80, which have been palletized for shipping. In yet another non-limiting embodiment, the syringes 1 may be individually packaged and sterilized before being placed into the container 80.

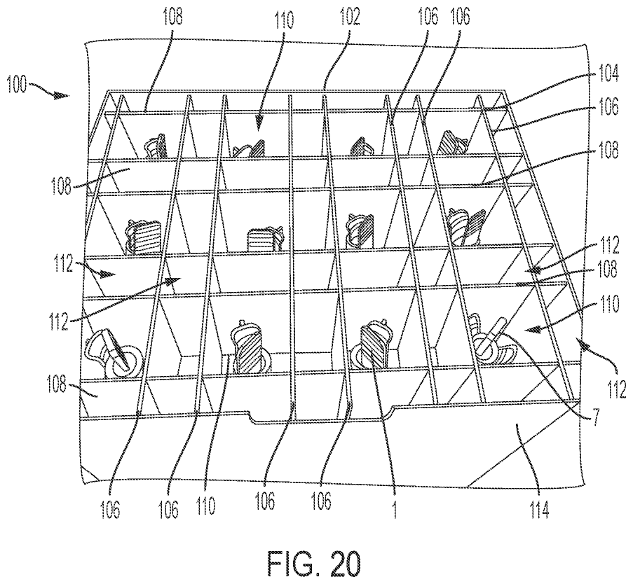

With reference to FIG. 20, a packaging system 100 according to another embodiment of the present disclosure is shown. The packaging system 100 includes a container 102 that may be shaped the same as container 12 or container 80, described herein in connection with FIGS. 1-19. The container 102 includes sidewalls 114, a bottoms surface 116, and a lid (not shown). According to various embodiments, the packaging system 100 may include a dividing wall arrangement 104 that differs from dividing wall arrangement 14, described herein. The packaging system 100 includes a dividing wall arrangement 104 that has a plurality of longitudinal walls 106 and a plurality of lateral walls 108. The longitudinal walls 106 and lateral walls 108 may have respective slots and pegs for assembly, or the walls 106, 108 may be formed as one piece. The walls 106, 108 define receiving spaces 110 for holding the syringes 1 and may also include secondary spaces 112 for isolating each syringe 1 and each syringe receiving space 110 from one another. As shown in FIG. 20, walls 106, 108 are shaped so that the syringes are held upright entirely within a receiving space 110. This means that in certain embodiments a cover may be omitted. The walls 106, 108 also define secondary spaces 112 that are not shaped to receive a syringe 1. As shown, the secondary spaces 112 may have a smaller volume relative to the syringe receiving spaces 110 and have a smaller surface area and volume than the receiving spaces 110. It is noted that other shapes and sizes of the secondary spaces 112 may be used in other non-limiting embodiments without deviating from the scope of the disclosure. As shown, the secondary spaces 112 are arranged to surround an individual receiving space 110. This arrangement allows the syringe 1 to move within its respective receiving space 110 and contact one or more of the lateral 106 and/or longitudinal walls 108 during transport but have a buffering space between adjacently packaged syringes 1. The presence of the secondary spaces 112 make it so that only one syringe 1 can contact any one wall 106, 108 at any one time. In other words, by not having syringes 1 located in adjacent receiving spaces 110, there is no risk of having the syringes becoming damaged because of contact against a common wall 106, 108 which may allow through contact between the bodies 2 of adjacently packaged syringes 1 through the common wall. The presence of the secondary spaces 112 also allows for some cushioning and collapsing volume to occur should a container 102 become compressed or damaged during transport. For clarity, only some of the syringes 1, longitudinal walls 106, lateral walls 108, receiving spaces 110, and secondary spaces 112 are labeled in FIG. 20.

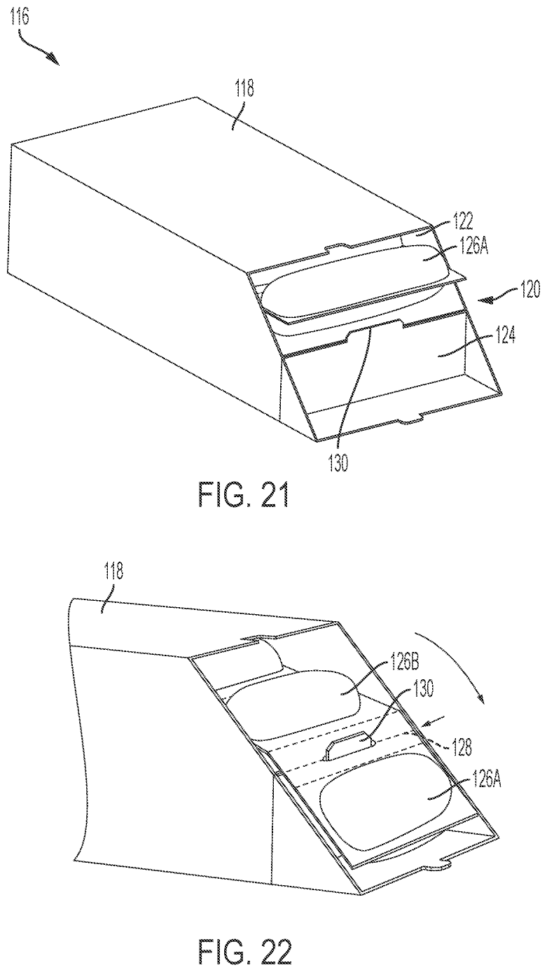

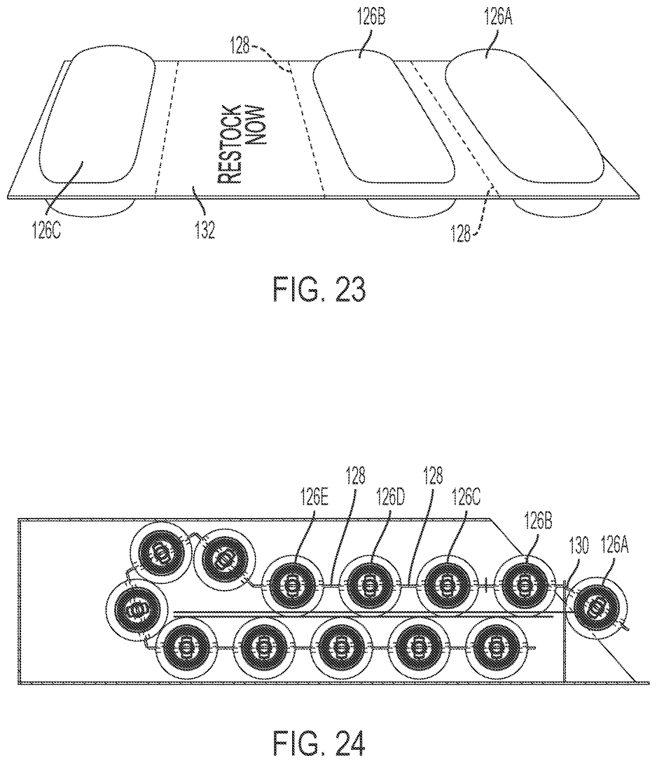

With reference to FIGS. 21-24, another packaging system 116 according to another non-limiting embodiment of the present disclosure is shown. The packaging system 116 is in the form of a shelf dispenser for container cartons 126 for dispensing individually packaged syringes 1 packaged in a series of separate packaging configurations. The packaging system 116 includes a container 118 having a front face 120 with an open portion 122 and a closed portion 124. The syringe container cartons 126 are removed via the open portion 122 via tearing engagement between perforations 128 between the individually packaged syringes 1 and lip 130 that extends from the closed portion 124. Multiple individual syringe container cartons 126A, 126B, 126C ... 126n are connected to each other in a strip that may be folded over itself within the container 118 to reduce overall size of packaging as illustrated in FIGS. 23 and 24. As shown in FIG. 24, a plurality of syringe container cartons 126 are included in the strip, however other quantities are contemplated depending on available shelf space, single versus dual syringe injector configurations, and the like. To remove an individual syringe container carton 126A from the strip, the container carton 126A is pulled outward and downward so that the lip 130 engages with the perforations 128 making it easy for the container carton 126A to be tearably removed from the strip. This process can be repeated for the total number of syringe container cartons 126 within the strip. As shown in FIG. 23, in certain embodiments a spacer 132 can be included in the strip between syringe container cartons located at the back end of the strip, so that a user knows when the container 118 is almost out of syringe container cartons 126 and should be replaced by a new container 118. As shown, the spacer 132 may include the indicator “RESTOCK NOW” or other suitable indicia to indicate that the number of syringe container cartons 126 remaining is low. The spacer 132 has perforations 128 to separate it from the remaining cartons 126. The spacer 132 may be placed before the final syringe container carton 126 within the strip or it may be placed before the final two or final three syringe container cartons 126 as desired. Each syringe container carton 126 may hold an individual syringe or other medical device. The cartons 126 may be made of recyclable material or biodegradable packaging material to lessen the impact on the environment when they are discarded. The syringe container cartons 126 may be sterilized individually and placed in the container 118 or they may be sterilized while inside of the container 118 as described herein.

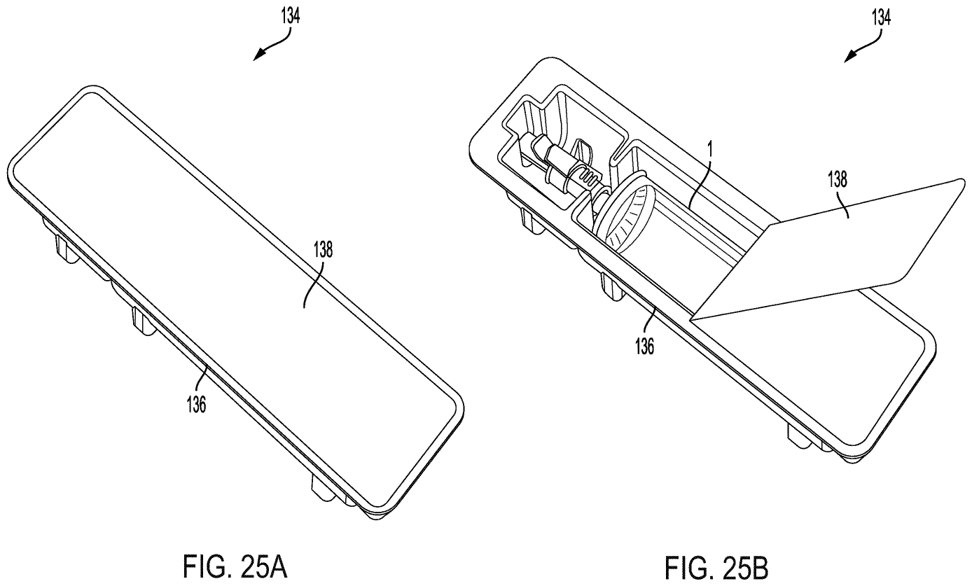

With reference to FIGS. 25A and 25B, an example of a single syringe sterile fluid path packaging configuration 134 is shown according to another embodiment of the present disclosure. The packaging may include a thermoformed plastic tray 136 with a peel-back cover 138 provided across an opening of the tray 136 to access the syringe 1. The tray 136 is shaped to include flanges and other molded features to fit the features of the syringe 1 snugly therein and minimize movement during shipping and handling. If different medical devices are included, the tray 136 may be formed to snugly fit those features in addition to those of syringe 1. The tray 136 protects the syringe during shipping and storage. In certain embodiments the thermoformed tray may further include one or more indexing features formed into outside of the tray surface. The indexing features may be configured to allow stacking of a plurality of the packaging 134 during packaging, shipping, and storage without damage or scratching of the syringes. After removal of the syringe 1, the thermoformed trays 136 may be stacked within each other, minimizing storage space for the used packaging materials until they can be removed, and disposed of or recycled. According to various embodiments, the peel-back cover 138 may be adhered to the tray 136 and may be manufactured from any suitable materials, such as Tyvek, nylon, other suitable medical packaging material, and the like, that is stable to sterilization techniques, as described herein, and allow ready removal of the contents of the package after peeling back the cover 138.

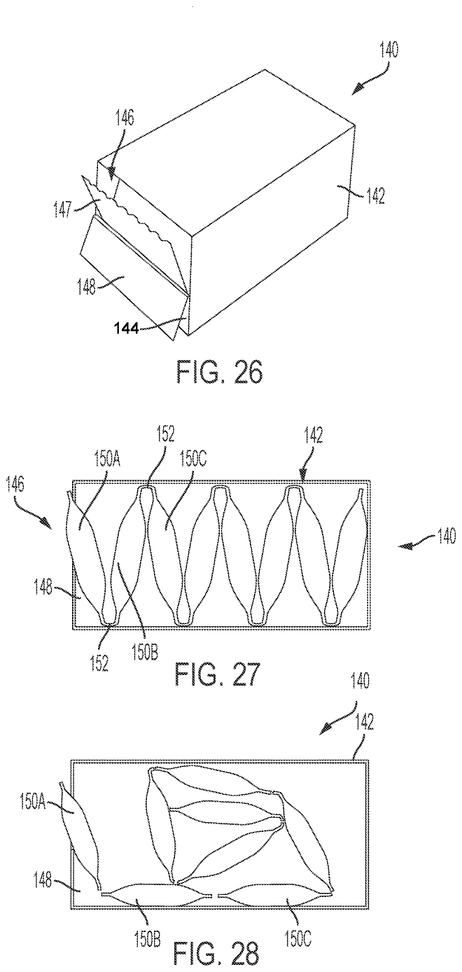

With reference to FIGS. 26-28, embodiments of another packaging configuration 140 for other fluid path components 150 such as connectors and/or tubing components is shown. The packaging configuration 140 includes a container 142 with a front face 144 having an opening portion 146 and a closed portion 148. The opening portion 146 may include a sidewall that is torn away from the front face 144 to access individually wrapped fluid path components 150. The individually wrapped components 150 may be in a sterile packaging or utilize a sterile fluid path configuration. As shown in FIG. 27, the individually wrapped components 150A, 150B, 150C ... 150n may be arranged in a strip and separated by perforations or folds 152 that allow for an individual component 150A to be easily separated from the strip. The perforations 152 are only for separating an individual component 150A from the strip and are not intended to open any one component to the atmosphere prior to use. The strip may be arranged in a roll or zig-zag configuration (as shown) within the container 142. In another embodiment, shown in FIG. 28, the individually wrapped components 150A, 150B, 150C ... 150n may be individually placed within the container 142, so that a single component 150A may be removed when needed, without needing to separate the component 150A from a strip. In certain embodiments, the individual packaging may include a separate opening mechanism, such as a separate set of perforations or a peel-back configuration to access the packaged fluid path component therein once it is removed from the container 142. In certain embodiments, the containers 142 of the individually wrapped components 150 necessary for setting up the fluid injection procedure may be stored on a shelf in a radiology suite so that they are readily accessible by the technician. The containers 142 may be designed to sit on the shelf and dispense the individually wrapped components 150 according to order of use during the set-up procedure. For example, the individual containers 142 may include indicia, such as numbers or alphabetical lettering, to indicate the order of access for set-up so that the technician can arrange the containers 142 on the shelf in the most convenient order for dispensing and set-up. According to certain embodiments, the containers 142 may include an opening for accessing the individually wrapped components 150, for example an access drawer near the bottom of the front of the shelf-top box that allows a gravity feed of the packaged fluid path components to the access point. In other embodiments, such as where the individually wrapped components 150 are packaged as a strip in a rolled or zig-zag configuration, the container 142 may allow access and removal of the individual components 150, for example from an unrollable roll configuration or configuration where zig-zag configured strip allows access to a single outer component 150 for ready disconnection from a following component by separation at a perforated portion.

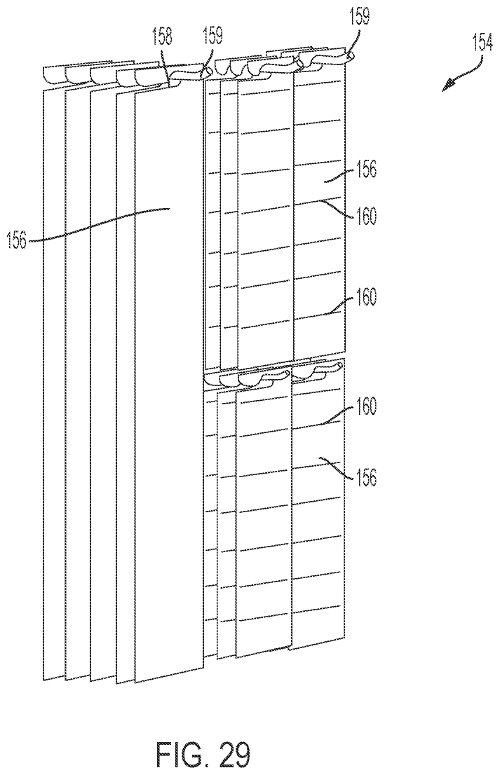

With reference to FIG. 29, according to other embodiments a hanging packaging configuration 154 may be used for ready access to individual fluid path components. Individually wrapped components 156 may be hung via a hook hole 158 from a hook 159 provided on a wall, shelf or other fixture. Other slots or holes may be arranged on the components 156 to facilitate hanging as well. The hook hole 158 may be formed on the packaging 156 without interfering with or contaminating the component wrapped therein. In other embodiments, the component may include a hook and the packaging 156 may naturally form a hooked shape around the hook of the component to facilitate hanging. In one embodiment, the individually wrapped component 156 may be individually hung on the hook 159, for example when the component 156 includes a long fluid path. In another embodiment, multiple individually wrapped components 156 may be provided in a strip and be separated by tearable perforations 160. As with the other perforations, these perforations 160 are arranged to only separate one component 156 from another and do not serve to open the packaging of any one component 156. In certain embodiments, the individual packaging 156 may include a separate opening mechanism, such as a separate set of perforations or a peel-back configuration to access the packaged fluid path component therein.

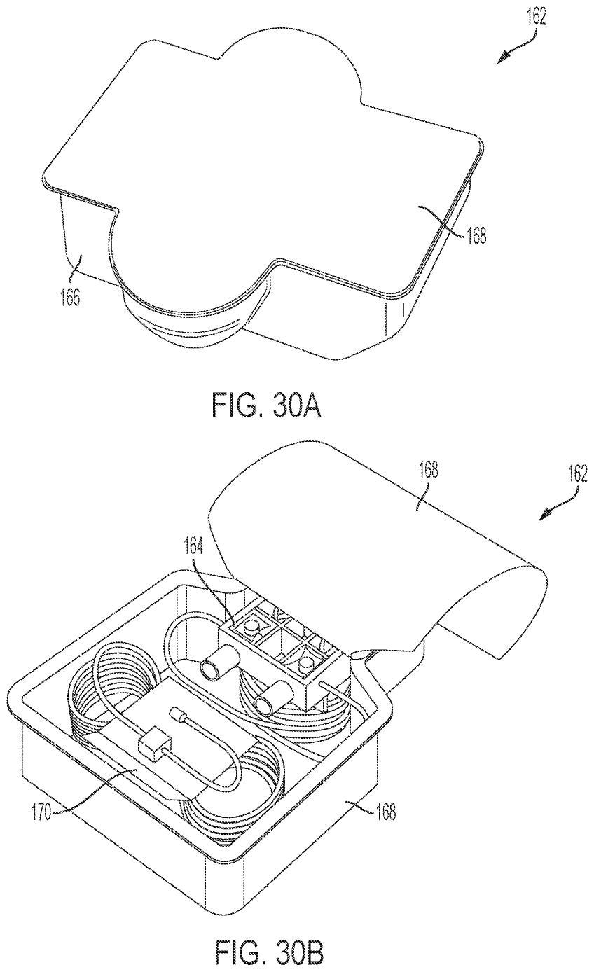

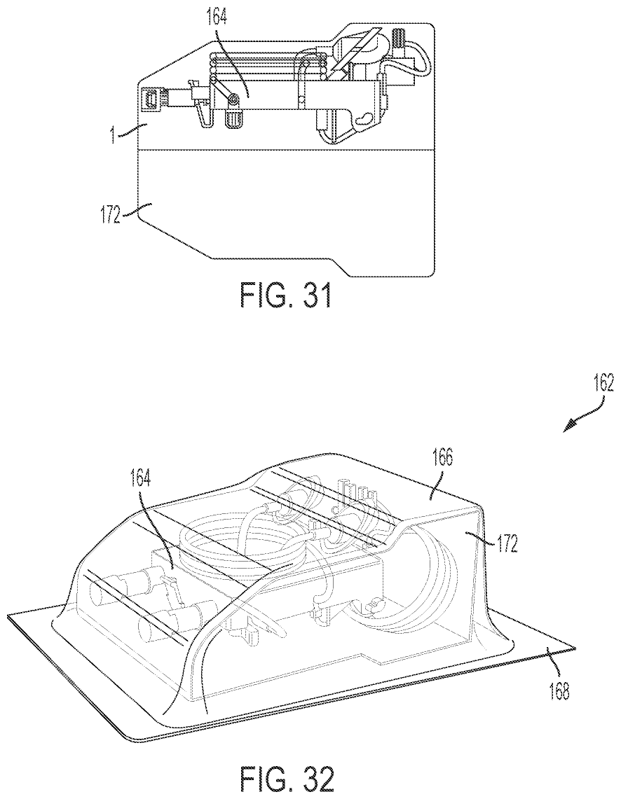

With reference to FIGS. 30A-32, a packaging configuration 162 for a multi-patient cassette 164 is shown. Examples of the cassette 164 are shown and described in International Patent Application Publication No. WO 2022/119837, the disclosure of which is hereby incorporated by reference in its entirety. The packaging configuration 162 includes a preformed or molded tray 166 with a peel-back cover or lid 168 as described herein. The tray 166 may include various recyclable or post-consumer content formed inserts 170 that act as retention and engagement features to hold and maintain the cassette 164 and fluid path components to prevent inadvertent movement and damage during packaging and shipping, while the formed shape of the tray 166 provides structural elements for protection and managing the tubing during shipping and storage. The peel-off lid 168 may be a polymeric formed material, such as Tyvek™, to maintain a sterile barrier in the case of a non-sterile fluid path or a non-sterile barrier in embodiments incorporating sterile fluid path elements. The inserts 170 may be made of cardstock, such as solid bleached sulfate (SBS) paperboard, to provide a means to manage the tubing during packaging, shipping, and storage to prevent damage or tangling of the tubing within the tray 166. The materials of the inserts 170 may be recyclable or readily biodegradable to reduce landfill and disposal costs. As shown in FIGS. 31 and 32, the packaging configuration 162 may also include sidewalls 172 that provide structure to the tray 166 and protection for the cassette 164. The sidewalls 172 may be made of cardboard, card stock, SBS paperboard, or similar materials as described herien. Additional structural elements may enable secure stacking during bulk packaging and storage to maximize packing quantities and/or minimizing shifting and movement during shipping.

Other embodiments of the present disclosure may include methods for packaging and sterilizing a plurality of syringes 1 or other fluid path components contained in a packaging system according to the various embodiments described herein. According to various embodiments, the methods may include packaging the plurality of syringes 1 in a syringe packaging system as described according to embodiments herein and irradiating at least a portion of the syringe packaging system with a sterilizing irradiation beam oriented in a direction substantially perpendicular to the longitudinal axis of the plurality of syringes. In certain embodiments the sterilizing irradiation beam may include an electron-beam sterilizing irradiation beam. As described herein according to certain embodiments, the plurality of syringes may be packaged in a staggered configuration such that a surface of the plunger of the at least one of the plurality of syringes receives an irradiation amount of the sterilizing irradiation beam that is substantially similar to an irradiation amount of the sterilizing irradiation beam received by a surface of the at least another one of the plurality of syringes. According to various embodiments, the plurality of syringes 1, may be packaged utilizing a sterile fluid path configuration.

While specific embodiments of the device of the present disclosure have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the device of the present disclosure which is to be given the full breadth of the claims appended and any and all equivalents thereof.

1. A syringe packaging system comprising:
a dividing wall arrangement defining a plurality of spaces;
a cover defining a plurality of openings, wherein each opening of the plurality of openings is configured to receive a distal end of a syringe of the plurality of syringes therethrough; and
a container configured to receive the dividing wall arrangement and a plurality of syringes therein,
wherein at least a portion of the plurality of spaces are configured to receive an individual syringe of the plurality of syringes therein, wherein the plurality of syringes are received in an upright, substantially parallel configuration and the dividing wall arrangement divides each individual syringe from one or more adjacent syringes.

2. (canceled)

3. The syringe packaging system of claim 1, wherein each of the plurality of openings comprises at least one slit circumferentially arranged around a periphery of each of the plurality of openings.

4. The syringe packaging system of claim 3, wherein each of the plurality of syringes comprises a clip feature located on the distal end, and wherein each of the plurality of openings are configured to receive the clip feature therethrough.

5. They syringe packaging system of claim 1, wherein the cover comprises a body and a plurality of barriers extending vertically upwards from the body, the body defining the plurality of openings, wherein the plurality of openings are arranged in a plurality of rows along the body, and wherein the each of the plurality of barriers extend adjacent to at least one row of the plurality of rows of openings.

6. The syringe packaging system of claim 5, wherein at least one first barrier of the plurality of barriers extends along an edge of the body and extends a first distance away from the body and at least one second barrier extends along an interior of the body and extends a second distance away from the body, and wherein the second distance is longer than the first distance.

7. The syringe packaging system of claim 1, wherein the dividing wall arrangement comprises a plurality of longitudinal walls and a plurality of lateral walls intersecting the plurality of longitudinal walls to define the plurality of spaces therebetween, and wherein the cover is configured to rest on a top surface of the dividing wall arrangement in the container.

8. The syringe packaging system of claim 1, wherein an internal bottom surface of the container is configured to support a proximal end of each of the plurality of syringes, and wherein at least a portion of the internal bottom surface is slanted at an angle relative to an outer bottom surface of the container such that the proximal end of at least one of the plurality of syringes is at a different elevation of at least another one of the plurality of syringes.

9. The syringe packaging system of claim 8, wherein a plunger of the at least one of the plurality of syringes is at least partially offset in a lateral direction from a plunger of the at least another one of the plurality of syringes.

10. The syringe packaging system of claim 8, wherein the internal bottom surface comprises a first slanted portion extending in a first direction and a second slanted portion extending in a second direction different from the first direction.

11. The syringe packaging system of claim 10, wherein the first slanted portion extends from a first end of the container and the second slanted portion extends from a second end of the container opposite the first end and wherein the first slanted portion and the second slanted portion meet to define a peak.

12. (canceled)

13. The syringe packaging system of claim 1, wherein a first portion of the plurality of spaces are configured to receive an individual syringe of the plurality of syringes therein and a second portion of the plurality of spaces are configured to not receive an individual syringe therein and wherein the second portion of the plurality of spaces are arranged to surround the first portion of the plurality of spaces.

14. (canceled)

15. The syringe packaging system of claim 13, wherein the second portion of the plurality of spaces has a volume that is less than a volume of the first portion of the plurality of spaces.

16. (canceled)

17. A packaging system for the sterilization of syringes, the system comprising:
a plurality of syringes, wherein each syringe of the plurality of syringe comprises:
a body defining a proximal end and a distal end;
a plunger disposed within the body proximate the proximal end; and
a tip at the distal end;
a dividing wall arrangement defining a plurality of spaces, at least a portion of the plurality of spaces being configured to receive one of the plurality of syringes therein, wherein each of the plurality of syringes are received in an upright, substantially parallel configuration and the dividing wall arrangement divides each individual syringe from one or more adjacent syringes;
a cover configured to receive the distal ends of the plurality of syringes therethrough; and
a container configured to receive the plurality of syringes, the dividing wall arrangement, and the cover therein,
wherein the plurality of syringes, dividing wall arrangement, and cover are received within the container such that each of the plurality of syringes are configured to be sterilized within the container by an outside source.

18. The packaging system of claim 17, wherein an internal bottom surface of the container is configured to support the proximal end of the syringes, and wherein at least a portion of the internal bottom surface is slanted such that the proximal end of at least one of the plurality of syringes is at a different elevation of at least another one of the plurality of syringes.

19. The syringe packaging system of claim 18, wherein a plunger of the at least one of the plurality of syringes is at least partially offset in a lateral direction from a plunger of the at least another one of the plurality of syringes.

20. (canceled)

21. The packaging system of claim 17, wherein the cover comprises a body defining a plurality of openings, wherein each of the plurality of openings is configured to receive the distal end of a syringe of the plurality of syringes therethrough.

22. The packaging system of claim 21, wherein each of the plurality of syringes further comprises a clip feature connected to the distal end, and wherein each of the plurality of openings are further configured to receive the clip feature therethrough.

23. The packaging system of claim 21, wherein the cover further comprises at least one barrier extending vertically upward from the body, the at least one barrier separating at least a first portion of the plurality of openings from at least a second portion of the plurality of openings.

24. (canceled)

25. A method for sterilizing a plurality of syringes contained in a packaging system, the method comprising:
packaging the plurality of syringes in a syringe packaging system, the syringe packaging system comprising:
a dividing wall arrangement defining a plurality of spaces;
a container configured to receive the dividing wall arrangement and a plurality of syringes therein; and
an internal bottom surface of the container configured to support a proximal end of the plurality of syringes, wherein at least a portion of the plurality of spaces are configured to receive an individual syringe of the plurality of syringes therein, wherein the plurality of syringes are received in an upright, substantially parallel configuration and the dividing wall arrangement divides each individual syringe from one or more adjacent syringes, wherein at least a portion of the internal bottom surface is slanted at an angle relative to an outer bottom surface of the container such that the proximal end of at least one of the plurality of syringes is at a different elevation of at least another one of the plurality of syringes, and wherein a plunger of the at least one of the plurality of syringes is at least partially offset in a lateral direction from a plunger of the at least another one of the plurality of syringes; and
irradiating at least a portion of the syringe packaging system with a sterilizing irradiation beam oriented in a direction substantially perpendicular to the longitudinal axis of the plurality of syringes.

26. (canceled)

27. The method of claim 25, wherein a surface of the plunger of the at least one of the plurality of syringes receives an irradiation amount of the sterilizing irradiation beam that is substantially similar to an irradiation amount of the sterilizing irradiation beam received by a surface of the at least another one of the plurality of syringes.
